(19) United States
(12) Patent Application Publication
(10) Pub. No.: US 2004/0082953 A1
(43) Pub. Date: Apr. 29, 2004

(54) Cage for immobilization of the spine and for osteosynthesis, method of manufacturing a cage, method of implanting a cage, and method of removing a cage

(76) Inventor: Dominique Petit, Verton (FR)

Correspondence Address: BACHMAN & LAPOINTE, P.C., 900 CHAPEL STREET, SUITE 1201, NEW HAVEN, CT, 06510, US

(21) Appl. No.: 10/353,644

(22) Filed: Jan. 29, 2003

Related U.S. Application Data
(63) Continuation-in-part of application No. PCT/FR01/02419, filed on Jul. 24, 2001.

(30) Foreign Application Priority Data
Jul. 31, 2000 (FR) 00/10075

(51) Int. Cl. A61B 17/56

(52) U.S. Cl. 606/61

(57) ABSTRACT

The present invention relates to an intersomatic cage for immobilization of the spine, comprising a central hollow space intended to be filled with bone fragments, in which cage said hollow space is delimited by a helical structure composed of helical turns which are not axially contiguous, in order to allow said cage to be removed by unscrewing the helical structure.

The present invention also relates to a method of producing an intersomatic cage, to a method of implanting a cage, and to a method of removing a cage.

### FIELD OF THE INVENTION AND PRIOR ART

The present invention relates to an intersomatic cage for immobilization of the spine, of the type comprising a central hollow space intended to be filled with bone fragments in order to permit osteosynthesis, to a method of manufacturing an intersomatic cage, to a method of implanting an intersomatic cage, and to a method of removing an intersomatic cage.

The function of an intersomatic implant is to restore and maintain an intervertebral space and to obtain osseous fusion of the two adjacent vertebral bodies, this being called “osteosynthesis”. To obtain this osteosynthesis, the intersomatic implants are hollow and are provided with a multiplicity of holes of various shapes; the hollow space is intended to permit the insertion of one or more bone grafts, and the holes are intended to allow the grafts to develop in contact with the vertebral bodies so as to obtain a solid and lasting osseous fusion of these same vertebral bodies.

In the prior art, intersomatic cages are already known comprising a central hollow space intended to be filled with bone fragments, in particular U.S. Pat. No. 4,961,740.

In that document, the intersomatic cage is formed by a steel cylinder in which eight holes uniformly spaced in the axial direction have been drilled, each hole being centered on a circle concentric with the axis of the cylinder, and in which one large hole has been drilled which is centered on the axis and has a radius substantially identical to that of the circle mentioned above. An S-shape is also formed in the outer surface of the cylinder, thus creating a succession of elevations and hollows, each intersection of a hollow with one of the eight holes thus forming a perforation. Moreover, the inner surface of the cylinder comprises a screw thread.

This hollow, cylinder-shaped intersomatic cage can thus be screwed into a bone cavity having a screw thread matching the V-shape.

When the central hollow space of the intersomatic cage is filled with bone grafts, osseous fusion then develops between the bone cavity and the grafts, by virtue of the perforations.

The prior art also includes U.S. Pat. No. 5,263,953 which relates to a cage for osseous fusion, having a helical structure composed of helical turns which are not axially contiguous. At its two ends, the helical structure terminates in radial sections which are oriented perpendicular to the axis of the helical structure and which are intended, both of them, to cooperate with grooves formed on the instrument used for the implantation. These two radial sections make it possible to prestress the helical structure in such a way that the diameter of this structure decreases in order to allow the helical structure to be implanted. The purpose of the helical structure is, therefore, to permit a radial expansion after implantation. The cage is therefore not screwed into the bone cavity.

The major disadvantage of the intersomatic cages of the prior art is that they cannot subsequently be removed without damaging the osseous fusion which has developed inside and through them.

This takes on great importance if ablation, that is to say removal, of the intersomatic cage is considered. The only solution then is to break up some of the osseous fusion which has taken place, so as to free the cage. This operation causes damage to the osseous fusion which has important consequences for the patient.

### SUMMARY OF THE INVENTION

The object of the present invention is to remedy the disadvantages of the prior art by making available an intersomatic cage which can be easily withdrawn after osseous fusion, without at all damaging the fusion which has developed. Thus, the osseous fusion can continue to completion in the space previously occupied by the structure of the cage, and the bone rapidly recovers its initial dimensions.

To do this, the present invention is of the type described above, and in its widest sense it is distinguished by the fact that said hollow space is delimited by a helical structure composed of helical turns which are not axially contiguous, in order to allow said cage to be removed by unscrewing the helical structure.

A helical turn extends in a winding of substantially 360°, not closed at its center, said winding having a central cavity.

The helical structure according to the invention is thus preferably made up of several turns in order to ensure good immobilization, and these turns are not contiguous axially, that is to say a space is formed between each of the successive turns in the direction of the axis of the helical structure, so as to permit osseous fusion between the grafts, the bone cavity and the adjacent vertebral bodies, by way of the free space between the turns.

Thus, the intersomatic cage according to the invention can be removed at any time, by unscrewing the helical structure composed of the helical turns.

The cross section of said helical turns can have a circular or oblong shape, or a pentagon shape with a rectangular or square base, or a heptagon shape consisting of a juxtaposed rectangle and triangle; a square shape being considered as a particular rectangular shape.

The intersomatic cage according to the invention preferably has a progressively decreasing cross section at a distal axial end, without any change in the general shape of the helical structure.

The intersomatic cage according to the invention preferably has, at a distal end, a cutting edge which is perpendicular to the axis of said helical structure.

The intersomatic cage according to the invention preferably comprises, at a proximal axial end, a gripping means or a means for connection to at least one other implant.

The present invention also relates to a method of manufacturing an intersomatic cage for immobilization of the spine, comprising a central hollow space intended to be filled with bone fragments, said hollow space being delimited by a helical structure composed of helical turns which are not axially contiguous, in which method said intersomatic cage is obtained by hollowing out the central core of a screw.

The present invention also relates to a method of implanting an intersomatic cage for immobilization of the spine, comprising a central hollow space intended to be filled with bone fragments, said hollow space being delimited by a helical structure composed of helical turns which are not axially contiguous, in which method a boring and tapping of the underlying and/or overlying osseous walls is first carried out, the tapping being carried out with the aid of a helical structure with a general shape identical to that of said cage, and in which method said helical structure is then screwed into the internal thread thus formed.

In an alternative form of the method of implanting the intersomatic cage for immobilization of the spine, a boring of the underlying and/or overlying osseous walls is first carried out and said helical structure is then screwed into the underlying and/or overlying osseous walls, without first tapping said osseous walls.

In another alternative form of the method of implanting the intersomatic cage for immobilization of the spine, said helical structure is screwed directly into the underlying and/or overlying osseous walls, without first tapping or boring said osseous walls.

The present invention also relates to a method of removing an intersomatic cage for immobilization of the spine, comprising a central hollow space intended to be filled with bone fragments, said hollow space being delimited by a helical structure composed of helical turns, which are not axially contiguous, in which method said helical structure is unscrewed.

The present invention has the advantage that it is very easy to remove the osseous fusion cage, without damaging the fusion which has developed, simply by unscrewing the helical structure.

The present invention also has the advantage that it is very easy to implant the osseous fusion cage, simply by screwing the helical structure.

No other element is provided inside the helical structure, so that these screwing and unscrewing operations are not impeded.

In addition, the helical structure can be self-cutting, that is to say it is able to form, at least partially or even completely in the wall of the vertebrae, the internal thread into which the helical structure is introduced.

### BRIEF DESCRIPTION OF THE DRAWINGS

The invention will be better understood from the following description of an embodiment of the invention, this description being given purely by way of explanation and with reference to the attached figures in which:

FIG. 1 illustrates a perspective view of an intersomatic cage according to the invention;

FIG. 2 illustrates a perspective view of an intersomatic cage according to the invention, comprising a gripping plate;

FIG. 3 illustrates a perspective view of an intersomatic cage according to the invention, which comprises a gripping plate and whose turns have a cross section of oblong shape;

FIG. 4 illustrates a perspective view of an intersomatic cage according to the invention which is produced by hollowing out the central core of a screw and whose turns have a cross section in the shape of a pentagon with a rectangular base;

FIG. 5 illustrates a front view of an intersomatic cage according to the invention whose turns have a cross section in the shape of a heptagon with a square base;

FIG. 6 illustrates a longitudinal cross section of the cage in FIG. 5;

FIG. 7 illustrates a perspective view of the cage in FIG. 5;

FIG. 8 illustrates a longitudinal cross section of an intersomatic cage according to the invention arranged in an intervertebral space;

FIG. 9 illustrates a longitudinal cross section of an intervertebral space which has been bored and internally threaded to receive an intersomatic cage according to the embodiment in FIGS. 5 and 6;

FIG. 10 illustrates a longitudinal cross section of the intervertebral space in FIG. 9 in which an intersomatic cage according to the embodiment in FIGS. 5 and 6 is introduced;

FIG. 11 illustrates a longitudinal cross section of an intervertebral space which has been bored to receive an intersomatic cage having a self-cutting helical structure;

FIG. 12 illustrates a longitudinal cross section of an intervertebral space which has been bored, to a lesser extent than that in FIG. 11, in order to receive an intersomatic cage having a self-cutting helical structure.

### DESCRIPTION OF THE PREFERRED EMBODIMENTS

The present invention concerns an intersomatic cage (1) for immobilization of the spine, illustrated in FIGS. 1 to 6 and 8. This intersomatic cage is intended to be implanted in a bone cavity and comprises a central hollow space (2) intended to be filled with bone grafts.

In the intersomatic cage (1) according to the invention, the hollow space (2) is delimited by a regular helical structure (3) composed exclusively of helical turns (4) which are not axially contiguous, that is to say which are separated by an identical pitch and do not touch one another in the direction of the axis A of said helical structure (3), in order to permit removal of said cage by unscrewing the helical structure, that is to say by turning the helical structure in the opposite direction of the pitch.

As the helical structure is unscrewed, the turns move in the hollow produced by the preceding turns in the osseous fusion and they cause no damage whatsoever to the osseous fusion which has developed between the turns.

The pitch of the helical structure can be oriented to the left or to the right; this is of no consequence.

The helical structures illustrated generally comprise five turns, but their number is not important; only the external dimensions of the helical structure are important. It would, for example, be quite conceivable to produce a helical structure comprising four and a half turns or six turns.

Thus, one or more bone grafts can be placed in said hollow space (2), and the osseous fusion between the bone cavity and the bone grafts takes place through the spaces (8) situated between the helical turns (4), and through the space at the distal axial end (5′), and possibly through the space at the proximal axial end (5).

The helical structure (3) is akin to that of a regular helical spring, although no quality of elastic return inherent to helical springs, for example following axial compression or axial extension, is made use of here. The helical structure, composed of several turns, is used only for its ability to be unscrewed once fusion has taken place, so as not to in any way damage this fusion upon ablation (removal) of the cage.

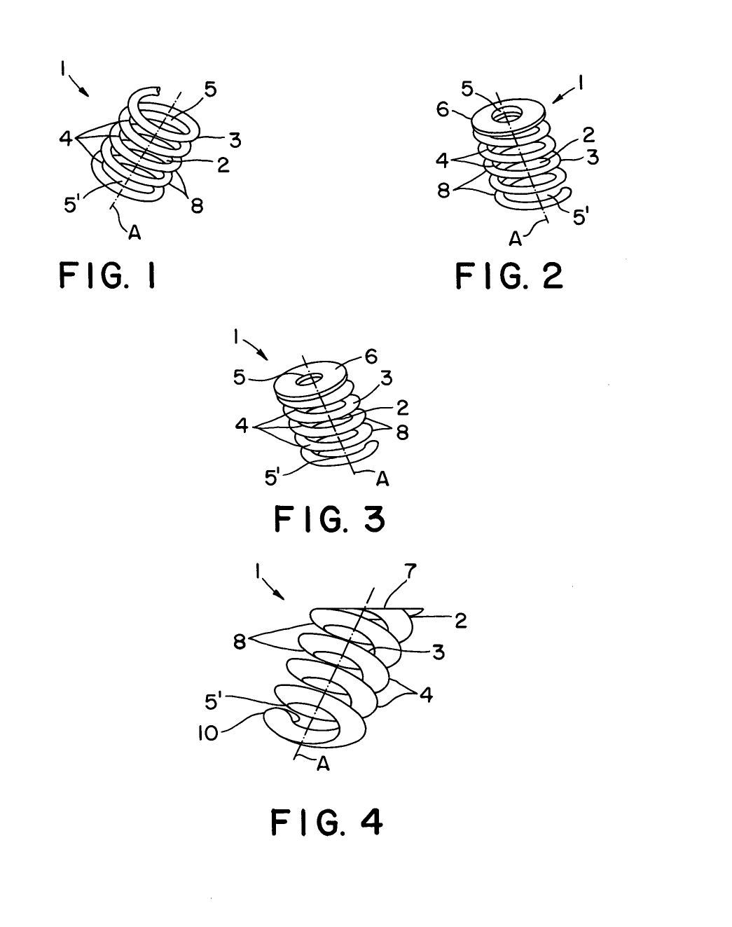

To make its implantation and its removal easier, the intersomatic cage (1) according to the invention comprises, preferably at its proximal axial end (5), a gripping means (6) and/or a means for connection to other implants, as is illustrated in FIGS. 2, 3, 5 to 7.

This gripping means (6) and/or means for connection to other implants is formed, for example, by a plate which has a threaded hole at its center, as is illustrated in FIGS. 2 and 3.

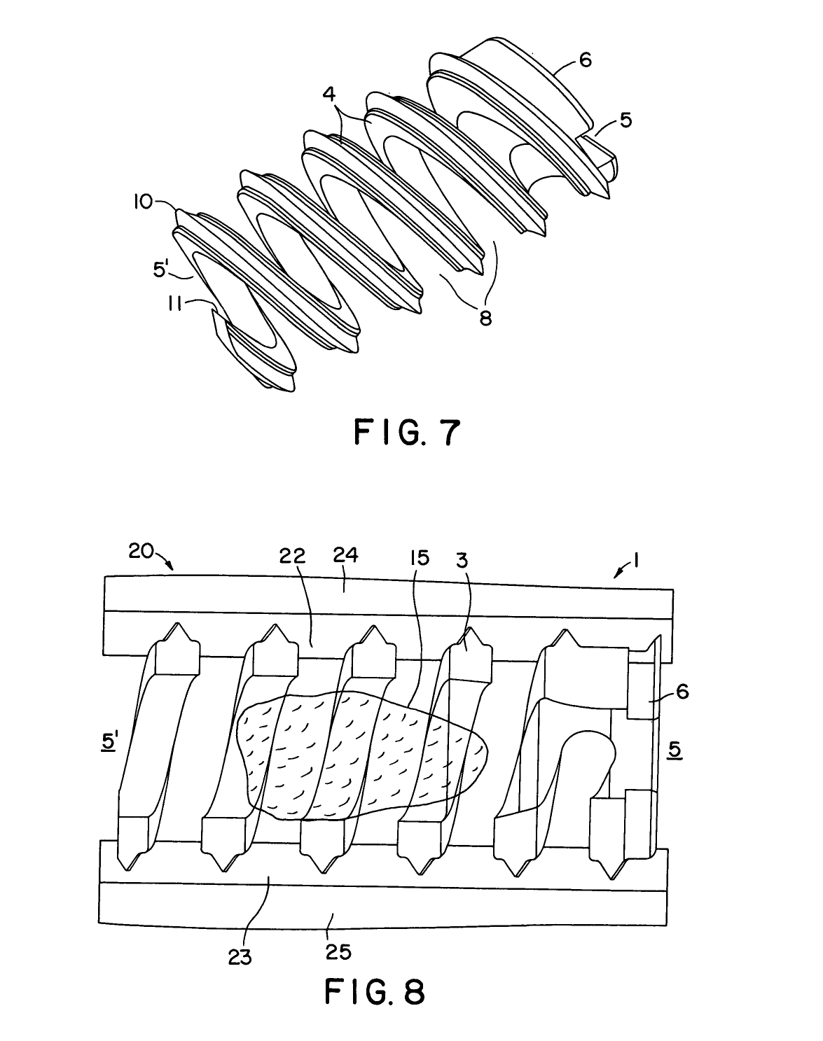

This gripping means (6) and/or means for connection to other implants can also be formed by a plate having a transverse notch, in the manner of a screw head, as is illustrated in FIGS. 5 to 7.

The distal axial end (5′) terminates in an abrupt cessation of its cross section or in a gradual reduction in its cross section. This gradual reduction in cross section creates a tip which may make screwing easier, but the shape of this tip is such that it at all times respects the helical shape of the helical structure (3). No element need be positioned at this end to permit screwing and unscrewing.

In cross section, said helical turns (4) have a shape facilitating the unscrewing of the intersomatic cage and, for example, have a circular shape, as illustrated in FIG. 1.

In cross section, said helical turns (4) can also have an oblong shape, as illustrated in FIG. 3, in order to improve immobilization by increasing the depth of anchoring of the intersomatic cage in the adjacent vertebral bodies in radial directions relative to the axis A.

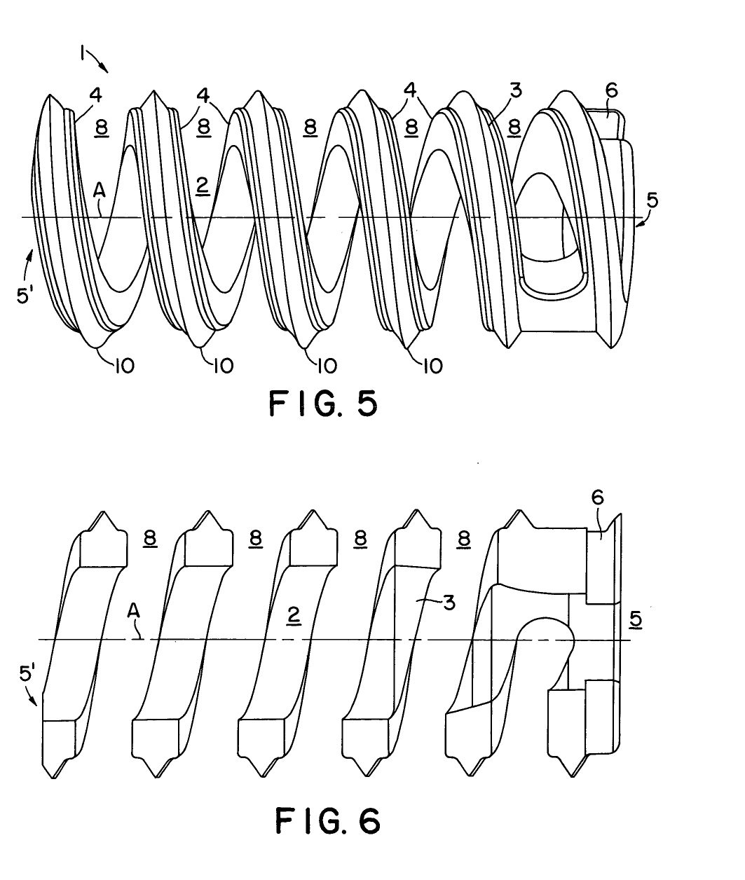

In cross section, said helical turns (4) can also have a pentagon shape with a rectangular or square base, the base being oriented toward the central hollow space (2), that is to say being centrifugal, as illustrated in FIG. 4.

This pentagon shape is thus formed by a rectangle and by a triangle which are mutually juxtaposed, each along one side. The base of the triangle in contact with the rectangle has a length identical to the length of that side of the rectangle against which the triangle is positioned. The triangle can be of any triangular shape.

The tip of the triangle directed away from the base forms a cutting edge (10) which facilitates implantation of the helical structure (3) by screwing and removal of the helical structure (3) by unscrewing.

In cross section, said helical turns (4) can also have a heptagon shape, as illustrated in FIGS. 5 and 6.

This heptagon shape is thus formed by a rectangular or square base and by a triangle which are mutually juxtaposed, each along one side.

The base of the triangle in contact with the rectangle has a length shorter than the length of that side of the rectangle against which the triangle is positioned. The triangle is preferably an isosceles triangle, or an equilateral triangle.

The tip of the triangle directed away from the base again forms a cutting edge (10) which facilitates implantation of the helical structure (3) by screwing and removal of the helical structure (3) by unscrewing.

In the embodiment in FIGS. 5 and 6, the outer triangular shape forms a threading which permits self-cutting of the adjacent vertebral wall.

It is important to note that the sides of the triangular shapes, both in the case of the pentagonal shape and also the heptagonal shape, are not necessarily straight. It is possible for one side of the triangle adjacent to the cutting edge (10) to be curved slightly in toward the inside of the triangle.

The present invention also relates to a method of producing an intersomatic cage (1), where said intersomatic cage (1) is obtained by hollowing out the central core of a screw (7), as illustrated in FIG. 4.

In this case, the cross section of said helical turns (4) has a pentagonal shape.

The screw can have a self-cutting thread.

An intersomatic cage (1) according to the invention can be produced easily and rapidly using this method, said intersomatic cage (1) also having means for its implantation and for its removal if, in addition, the head of the screw is at least partially retained.

FIG. 8 illustrates a vertebral structure (20) in which an intersomatic cage (1) according to the invention, and including a graft (15), is implanted.

To facilitate the implantation of the cage according to the invention, it may first be necessary to bore and tap the bone cavity in order to create, in the inner wall, a partially helical shape which exactly matches the shape of the helical structure (3). This operation is thus performed using a boring instrument and a tapping instrument.

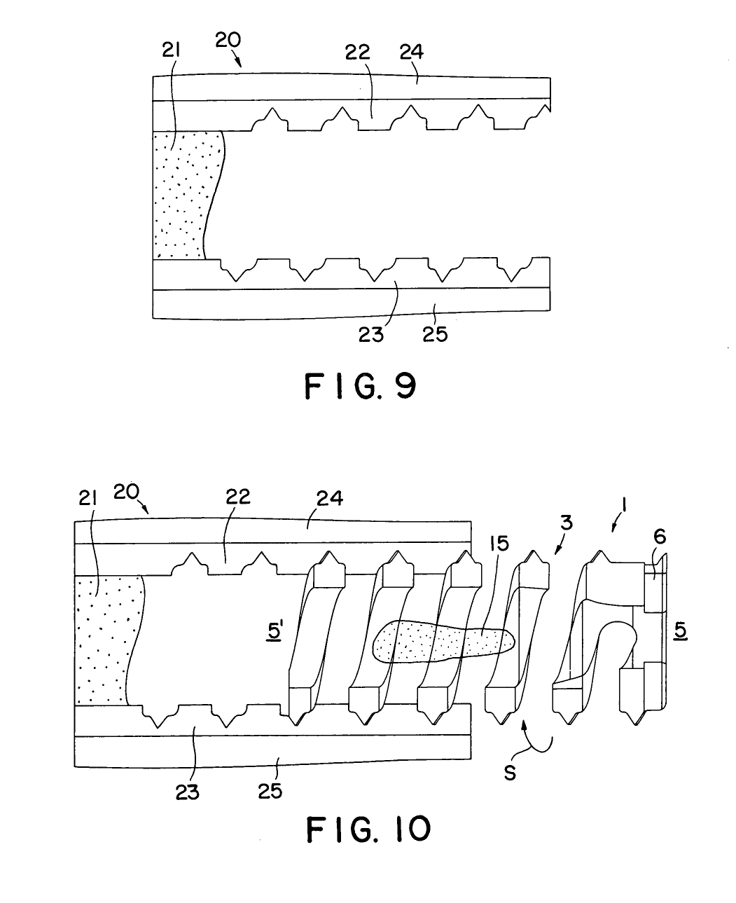

FIG. 9 illustrates a vertebral structure (20) composed of a central part (21) which corresponds to the disk and has been bored, and of overlying (22) and underlying (23) walls which correspond to the cortical bone and have been tapped. Reference numbers 24 and 25 illustrate the overlying and underlying spongy substance.

Once the tapping operation has been completed, the helical structure (3) of the prosthesis is screwed into the internal thread, as can be seen in FIG. 10.

It is also possible to use a self-cutting helical structure. This eliminates the step of initial tapping of the vertebral structure. The helical structure (3) of the prosthesis is then screwed directly into the vertebral structure.

FIG. 11 illustrates the screwing of a helical structure (3) into a vertebral structure (20) which has been tapped very wide, but not bored. In this case, the bore formed has a diameter identical to that of the centrifugal end of the rectangular shape of the heptagon.

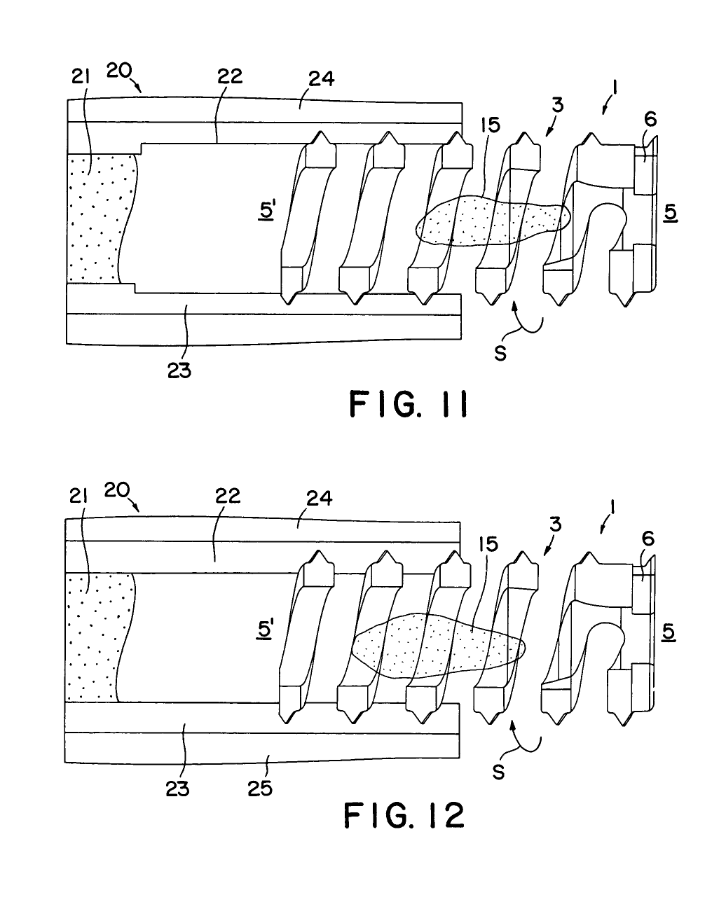

FIG. 12 illustrates the screwing of a helical structure (3) into a vertebral structure (20) which has been tapped only a little, and not bored. In this case, the bore formed has a diameter identical to between the centrifugal end of the rectangular shape of the heptagon and the centripetal end of the rectangular shape of the heptagon.

It is also possible to dispense with a tapping operation and to screw the helical structure (3) directly into the vertebral structure (20).

In FIGS. 10 to 12, the arrow S illustrates the direction of movement in which the helical structure (3) is screwed into the vertebral structure (20).

To remove the intersomatic cage, the helical structure of the prosthesis is simply unscrewed by turning the cage in the opposite direction of the arrow V.

The invention has been described above by way of illustration. It will be appreciated that the person skilled in the art will be able to produce different alternatives of the invention without in so doing departing from the scope of the patent.

1. An intersomatic cage for immobilization of the spine, comprising a central hollow space intended to be filled with bone fragments, in which cage said hollow space is delimited by a helical structure composed of helical turns which are not axially contiguous, in order to allow said cage to be removed by unscrewing the helical structure.

2. The intersomatic cage as claimed in claim 1, in which the cross section of said helical turns has a circular shape.

3. The intersomatic cage as claimed in claim 1, in which the cross section of said helical turns has an oblong shape.

4. The intersomatic cage as claimed in claim 1, in which the cross section of said helical turns has a pentagon shape with a rectangular base.

5. The intersomatic cage as claimed in claim 1, in which the cross section of said helical turns has a heptagon shape consisting of a juxtaposed rectangle and triangle.

6. The intersomatic cage as claimed in claim 1, having a progressively decreasing cross section at a distal axial end.

7. The intersomatic cage as claimed in claim 1, having, at a distal end, a cutting edge which is perpendicular to the axis of said helical structure.

8. The intersomatic cage as claimed in claim 1, having, at a proximal axial end, a gripping means.

9. The intersomatic cage as claimed in claim 1, having, at a proximal axial end, a means for connection to at least one other implant.

10. A method of manufacturing an intersomatic cage for immobilization of the spine, comprising a central hollow space intended to be filled with bone fragments, said hollow space being delimited by a helical structure composed of helical turns which are not axially contiguous, in which method said intersomatic cage is obtained by hollowing out the central core of a screw.

11. A method of manufacturing an intersomatic cage for immobilization of the spine, comprising a central hollow space intended to be filled with bone fragments, said hollow space being delimited by a helical structure composed of helical turns which are not axially contiguous, in which method said intersomatic cage is obtained by hollowing out the central core of a self-cutting screw.

12. A method of implanting an intersomatic cage for immobilization of the spine, comprising a central hollow space intended to be filled with bone fragments, said hollow space being delimited by a helical structure composed of helical turns which are not axially contiguous, in which method a boring and tapping of the underlying and/or overlying osseous walls is first carried out, the tapping being carried out with the aid of a helical structure with a general shape identical to that of said cage, and in which method said helical structure is then screwed into the internal thread thus formed.

13. A method of implanting an intersomatic cage for immobilization of the spine, comprising a central hollow space intended to be filled with bone fragments, said hollow space being delimited by a self-cutting helical structure composed of helical turns which are not axially contiguous, in which method a boring of the underlying and/or overlying osseous walls is first carried out, and in which method said helical structure is then screwed into the underlying and/or overlying osseous walls, without first tapping said osseous walls.

14. A method of implanting an intersomatic cage for immobilization of the spine, comprising a central hollow space intended to be filled with bone fragments, said hollow space being delimited by a self-cutting helical structure composed of helical turns which are not axially contiguous, in which method said helical structure is screwed directly into the underlying and/or overlying osseous walls, without first tapping and boring said osseous walls.

15. A method of removing an intersomatic cage for immobilization of the spine, comprising a central hollow space intended to be filled with bone fragments, said hollow space being delimited by a helical structure composed of helical turns, which are not axially contiguous, in which method said helical structure is unscrewed.
